(19) United States
(12) Patent Application Publication
(10) Pub. No.: US 2010/0087911 A1
(43) Pub. Date: Apr. 8, 2010

(54) IMPLANT WITH A BASE BODY OF A BIOCORRODIBLE MANGANESE ALLOY

(76) Inventor: Dr. Heinz Mueller, Erlangen (DE)

Correspondence Address: GREER, BURNS & CRAIN, 300 S WACKER DR, 25TH FLOOR, CHICAGO, IL, 60606, US

(21) Appl. No.: 12/570,345

(22) Filed: Sep. 30, 2009

(30) Foreign Application Priority Data
Oct. 2, 2008 (DE) 10 2008 042 578.8

(51) Int. Cl. A61F 2/06; C22C 30/00; C22C 22/00; C22C 38/04; C22C 5/04; A61B 17/08

(52) U.S. Cl. 623/1.15; 420/580; 420/581; 420/434; 420/82; 420/461; 606/151

(57) ABSTRACT

The invention relates to an implant with a base body composed entirely or in parts of a biocorrodible manganese alloy.

### FIELD OF THE INVENTION

The invention relates to an implant with a base body composed entirely or in parts of a biocorrodible manganese alloy.

### BACKGROUND OF THE INVENTION

Implants are used in a variety of embodiments in modern medical technology. Implants serve, among other things, to support blood vessels, hollow organs and duct systems (endovascular implants), to attach and temporarily secure tissue implants and tissue transplants, as well as for orthopedic purposes, for example, as nails, plates or screws.

The implantation of stents is one of the most effective therapeutic measures in the treatment of vascular diseases. The purpose of stents is to take on a support function in the hollow organs of a patient. Stents of a conventional design therefore have a filigree support structure of metallic struts, which is initially present in a compressed form for introduction into the body and is expanded at the site of the application. One of the main areas of application of stents of this type is for permanently or temporarily widening vascular constrictions, in particular, constrictions (stenoses) of the coronary vessels, and then keeping the constricted areas open. In addition, aneurysm stents are also known, for example, which serve to support damaged vascular walls.

The base body of every implant, in particular of stents, comprises an implant material. An implant material is a non-living material that is used for an application in medicine and interacts with biological systems. The basic prerequisites for the use of a material as an implant material, which is in contact with the body's environment when used as intended, is that the material must be compatible with the body (biocompatibility). Biocompatibility means the ability of a material to induce an appropriate tissue reaction in a specific application. This includes an adaptation of the chemical, physical, biological and morphological surface properties of an implant to the recipient tissue with the objective of obtaining a clinically desirable interaction. The biocompatibility of the implant material also depends on the chronological course of the reaction of the biosystem into which it is implanted. Irritation and inflammation, which may lead to tissue changes, thus occur relatively rapidly. Biological systems react in different ways depending on the properties of the implant material. Depending on the reaction of the biosystem, the implant materials may be subdivided into bioactive, bioinert and degradable/absorbable materials. For the purposes of the present invention only degradable/absorbable metallic implant materials are of interest, which is referred to below as biocorrodible metallic materials.

The use of biocorrodible metallic materials is recommended in particular because often the implant needs to remain in the body only temporarily to fulfill the medical purpose. Implants of permanent materials, that is, materials that are not degraded in the body, may have to be removed again, since rejection reactions of the body can occur in the medium term and long term, even when there is a high biocompatibility.

One approach for avoiding a further surgical procedure is therefore to make the implant entirely or in parts of a biocorrodible metallic material. Biocorrosion means processes that are caused by the presence of endogenous media and which lead to a gradual degradation of the structure made of the material. At a certain point in time the implant or at least the part of the implant that is made of the biocorrodible material loses its mechanical integrity. The degradation products are largely absorbed by the body. As in the case of magnesium, for example, in the best case the degradation products even have a positive therapeutic effect on the surrounding tissue. Small quantities of unabsorbable alloy constituents are tolerable as long as they are nontoxic.

Known biocorrodible metallic materials comprise pure iron and biocorrodible alloys of the main elements magnesium, iron, zinc, molybdenum and tungsten. It is proposed among other things in DE 197 31 021 A1 to make medical implants from a metallic material, the main constituent of which is an element from the group of alkali metals, alkaline earth metals, iron, zinc and aluminum. Alloys based on magnesium, iron and zinc are described as particularly suitable. Secondary constituents of the alloys can be manganese, cobalt, nickel, chromium, copper, cadmium, lead, tin, thorium, zirconium, silver, gold, palladium, platinum, silicon, calcium, lithium, aluminum, zinc and iron. Regardless of the advances that have been achieved in the field of biocorrodible metal alloys, the alloys known so far have only limited usability because of their corrosion behavior.

It has thus been shown that metallic implants of magnesium alloys in vivo already lose their support strength after only a few days due to the rapid dissolution.

The application possibilities are also limited for pure iron or known iron alloys because of the relatively slow biocorrosion.

Various alloy elements were tested in order to increase the corrosion of pure iron or iron alloys under physiological conditions. Thus, for example, manganese is suitable for this purpose, which is generally known as an alloy element for highly wear-resistant steels, so-called manganese steels, and is used there in quantities of approx. 12 to approx. 25% by weight. However, these alloys generally have only a slight corrosion tendency under the usual application conditions.

### SUMMARY OF THE INVENTION

The object of the present invention is therefore to provide a biocorrodible metallic material for an implant, which is improved with respect to its corrosion behavior. This is to be achieved in particular in that the further material properties that are important for processing, such as, for example, its ductility and strength, are not substantially changed and optionally are even improved.

The object is attained according to the invention through a medical implant, the base body of which is composed entirely or in parts of an alloy, the main constituent of which is manganese (manganese-based alloy).

### DETAILED DESCRIPTION OF THE INVENTION

The alloy according to the invention contains manganese as the main component. According to the invention, the main component of an alloy means the alloy component, the atomic fraction of which in the alloy is the highest. In the preferred alloy compositions described in even more detail below, all of the further fractions of the alloy components are to be provided such that respectively manganese including contaminants due to production has the highest atomic fraction. Accordingly, all of the percentages for the composition of the alloy are to be understood as data in atomic percent.

Elementary manganese is a steel white and hard heavy metal which exists in four different modifications with different crystallographic structure (α, β, γ, δ manganese) which are available when heated to 740° C., 1075° C. and to above 1140° C. Thus manganese crystallizes at temperatures below 720° C. as α-Mn, the modification of the element that is stable and very brittle at room temperature, which is present as body-centered cubic metal lattice (body-centered cubic phase, a phase).

Within the scope of the present invention, particularly preferred manganese-based alloys are γ-manganese alloys. These are characterized by the formation of a face-centered cubic metal lattice, a so-called γ-phase or austenitic phase.

The term austenite is used to characterize face-centered cubic steels, e.g., the commercially available CrNi steels, as well as for face-centered cubic modifications of other alloys, such as, e.g., with the Ni—Ti system.

An austenite within the scope of the present invention is a face-centered cubic mixed crystal of a manganese-based alloy, with which the body-centered cubic phase present with pure manganese at room temperature is suppressed by one or more alloying elements or by the combination of one or more alloying elements and a thermal treatment from the γ field. On the one hand, this can occur through certain concentrations of suitable alloying elements alone, through which the conversion from the γ field during cooling with the melt metallurgical production of the alloys does not occur. On the other hand, this can take place in that an alloy with an element that makes the conversion difficult, i.e., shifts to lower temperatures, but cannot suppress it completely, is chilled from the γ field to room temperature and is present there as a metastable γ phase. Suitable alloying elements in the above sense are, e.g., C, N, Pd, Pt, Rh, Ru, V, Ir, Cu, Fe and Au.

These austenitic alloys are paramagnetic and, depending on the choice of the or the other alloying elements, render possible a better workability and ductility than the brittle α- or β-manganese.

In the event that the γ phase of the manganese-based alloy is not stable at room temperature, one skilled in the art knows thermochemical methods, for example, through the adjustment of the cooling rate during the production of the alloy, for suppressing the conversion of the face-centered cubic phase into the α-phase or β-phase so that a purely austentic structure is present. The temperature is thereby reduced through the rapid cooling so far that the diffusion necessary for the phase transition can no longer take place and the γ phase is frozen at room temperature in a metastable manner.

Particularly good corrosion properties were observed for manganese alloys with iron and/or iridium as a further alloying element.

Manganese-iron alloys show particularly favorable corrosion properties for a biological degradation from a proportion of more than 50% manganese.

Particularly preferably the proportion of iron in the manganese alloy according to the invention is 40 to less than 50%. With this proportion of iron the austentic structure of the manganese alloys is thermodynamically stable up to room temperature.

With a proportion of iron in the manganese alloy of 50 to 60%, the manganese alloys are characterized by particularly advantageous degradation times. These manganese alloys are further characterized by a high wear resistance.

Furthermore preferably the proportion of iron is 5 to 40%. A substantial reduction of the corrosion resistance was measured in tests in particular with an alloying of 10 to 30% iron.

It has been shown that manganese alloys with iron, in particular γ-manganese alloys, solidify considerably during deformation and are extremely wear resistant.

Advantageous degradation times further result with materials with the main constituent of manganese and up to 25% iridium.

Particularly preferably the proportion of iridium in the manganese alloy according to the invention is between 8 and 15%. These manganese alloys show a considerably better ductility than the brittle α-phase of the manganese and a high strength. Also in this alloying range the γ phase of the manganese is thermodynamically stable up to room temperature.

With higher or lower alloying contents of iridium, the material has to be subjected to thermal treatment to obtain a face-centered cubic lattice stable at room temperature.

If iron as well as iridium is alloyed to pure manganese, the corrosion resistance can likewise be reduced. In tests a considerable reduction of the corrosion resistance was measured, e.g., in particular with an alloying between 30 and 40% iron with an iridium content of up to 10% or between 10 to 20% iron and with an iridium content between 8 and 20%.

Table 1 shows the particularly preferred alloying ranges in the ternary system Mn—Fe—Ir alloy. The data for the proportions of iron and iridium in the respective formulas (I) to (IV) are in atomic percent.

TABLE 1
Mn—Fe—Ir alloy

| | | |
|---|---|---|
| Mn—Fe_{(50-3.57X)}—Ir_{X} | Where X = 0.1-13.99 | Formula (I) |
| Mn—Fe_{(40-5.0X)}—Ir_{X} | Where X = 0.1-7.99 | Formula (II) |
| Mn—Fe_{(50-2.0X)}—Ir_{X} | Where X = 0.1-24.99 | Formula (III) |
| Mn—Fe_{(20-10.0X)}—Ir_{X} | Where X = 0.1-1.99 | Formula (IV) |

The biological, mechanical and chemical properties of the materials according to the invention can furthermore be positively influenced if one or more elements are provided selected from the group comprising copper (0-20 atomic %), gold (0-20 atomic %), palladium (0-20 atomic %), platinum (0-20 atomic %), rhenium (0-20 atomic %), ruthenium (0-10 atomic %), vanadium (0-10 atomic %), carbon (0-5 atomic %), nitrogen (0-5 atomic %), arsenic (0-5 atomic %) and selenium (0-5 atomic %).

The manganese alloys according to the invention are to be selected in their composition such that they are biocorrodible. Alloys are described as biocorrodible as defined by the invention with which a degradation/conversion takes place in a physiological environment so that the part of the implant made from the material is entirely or at least chiefly no longer present. Artificial plasma as specified for biocorrosion tests according to EN ISO 10993-15:2000 (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l) is used as the test medium for testing the corrosion behavior of an alloy in question. A sample of the alloy to be tested is to this end stored in a sealed sample container with a defined quantity of the test medium at 37° C. At intervals of a few hours to several months, based on the corrosion behavior to be expected, the samples are removed and examined for traces of corrosion in a known manner. The artificial plasma according to EN ISO 10993-15:2000 corresponds to a hematoid medium and thus represents a possibility of reproducibly adjusting a physiological environment as defined by the invention.

To set the time for which the implant is intended to retain its functionality, it is advantageous if the choice of the special alloy composition is coordinated with its special degradation kinetics and the dimensioning of the implant.

It is therefore desirable for the mechanical integrity of a stent in vivo to remain stable for between 2 and 6 months.

It should be noted that manganese is essential for all biological organisms. However, deficiency symptoms in humans are unknown and even high overdoses are well tolerated. The daily requirement is between 0.4 and 10 mg. In animal experiments manganese has been identified as an essential element for osteogenesis and chondrogenesis. Further animal tests with manganese deficiencies have shown deficits in insulin production, changes in the lipoprotein metabolism and disturbances in the metabolism of growth factors. Furthermore, manganese is possibly a necessary cofactor in the conversion of preprothrombin to prothrombin. Biochemical tests have further shown that manganese is a cofactor for a number of enzymes, including for arginase and the alkaline phosphatase of the liver and for pyruvate carboxylase. The activity of succinic dehydrogenase and prolidase as well as some enzymes of the mucopolysaccharide synthesis is also increased through manganese. Accordingly the release of low amounts of manganese in the body in the course of the degradation of the alloy is harmless from a toxicological point of view.

Implants as defined by the invention are devices introduced into the body by a surgical method or a minimally invasive procedure and comprise fastening elements for bones, for example, screws, plates or nails, surgical suture material, intestinal clamps, vascular clips, prostheses in the area of hard and soft tissue, for example, stents and anchoring elements for electrodes, in particular of pacemakers or defibrillators. The implant is composed entirely or in parts of the biocorrodible material.

Preferably the implant of the biocorrodible manganese alloy is a stent for blood vessels, bile duct, urethra, esophagus, etc.; i.e., a supporting or connecting implant for all vessels, duct systems or cavity connections in the human body. Stents of conventional design have a filigree structure of metallic struts, which is first in a non-expanded state for introduction into the body and then is widened into an expanded state at the site of application.

Furthermore, the implant of the biocorrodible manganese alloy is in particular a clip for closing severed blood vessels. For example, a v-shaped clip with which a severed vessel is closed in that the clip is squeezed/plastically deformed at the vessel end with forceps such that it closes the vessel end so that the blood flow comes to a stop and the blood clots.

Moreover the implant of the biocorrodible manganese alloy can be in particular an implant that is used to safely close again vessels into which a cannula or a catheter with larger diameter has been temporarily inserted into the vascular system, after the temporary implant has been removed in order to avoid hemorrhages at this point. In this case the implant typically has the shape of a clamp, which is implanted by means of an application system and in which claws or tips grip the vascular wall around the point to be closed and press the opening together.

Finally the implant of the biocorrodible manganese alloy is in particular an occluder, specifically a septal occluder. An occluder is a minimally invasive applicable fixing system with which, e.g., a septal defect (PFO) can be fixed until the cardiac septum grows together and the defect is naturally closed. Subsequently the implant can be biologically degraded. The implant is embodied thereby e.g., such that a long tubular structure is compressed and plastically deformed by a tensile force in front of and behind the defect so that on both sides of the defect a screen shape is formed which presses both parts of the open cardiac septum together.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. An implant with a base body comprising a biocorrodible metallic material, characterized in that the material is an alloy, the main component of which is manganese.

2. An implant according to claim 1, characterized in that the material is an alloy, the main component of which is γ-manganese.

3. An implant according to claim 1, characterized in that the material contains one or more of iron and iridium.

4. An implant according to claim 3, in which the proportion of iron in the manganese alloy is 0 to less than 50% (atomic).

5. An implant according to claim 4, in which the proportion of iron in the manganese alloy is 40 to 49% (atomic).

6. An implant according to claim 4, in which the proportion of iron in the manganese alloy is 5 to 40% (atomic).

7. An implant according to claim 3, in which the proportion of iridium in the manganese alloy is 0 to 25% (atomic).

8. An implant according to claim 3, in which the proportion of iron in the manganese alloy is between 30 and 40% (atomic) and the proportion of iridium in the manganese alloy is up to 10% (atomic).

9. An implant according to claim 3, in which the proportion of iron in the manganese alloy is between 10 and 20% (atomic) and the proportion of iridium in the manganese alloy is 8 to 20% (atomic).

10. An implant according to claim 3, in which the proportions of iron and iridium in the manganese alloy are according to
Mn—FE_{(50-3.57X)}—Ir_{X}, where X=0.1-13.99. Formula (I)

11. An implant according to claim 3, in which the proportions of iron and iridium in the manganese alloy are according to
Mn—FE_{(40-5.0X)}—IR_{X}, where X=0.1-7.99. Formula (II)

12. An implant according to claim 3, in which the proportions of iron and iridium in the manganese alloy are according to
Mn—Fe_{(50-2.0X)}—Ir_{X}, where X=0.1-24.99. Formula (III)

13. An implant according to claim 3, in which the proportions of iron and iridium in the manganese alloy are according to
Mn—Fe_{(20-10.0X)}—Ir_{X}, where X=0.1-1.99. Formula (IV)

14. An implant according to claim 1, in which the manganese alloy additionally contains one or more elements selected from the group comprising copper, gold, palladium, platinum, rhenium, ruthenium, vanadium, carbon, nitrogen, arsenic and selenium.

15. An implant according to claim 1, in which the implant is one or more of a stent, a clip, an occluder and an implant for temporarily securing tissue.

16. An implant according to claim 4, in which the proportion of iron in the manganese alloy is 10 to 30% (atomic).

17. An implant according to claim 3, in which the proportion of iridium in the manganese alloy is and preferably 8 to 15% (atomic).

18. An implant for implanting in a human for medical purposes, the implant comprising: a base body comprising a biocorrodible metal alloy, manganese being the highest atomic fractional component of the alloy, the alloy further comprising from about 30% to about 40% (atomic) iron and up to about 10% (atomic) iridium, the alloy further comprising up to 20% (atomic) of one or more elements selected from the group comprising copper, gold, palladium, platinum, rhenium, the alloy further comprising up to 10% (atomic) of one or more elements selected from the group comprising ruthenium and vanadium, the alloy further comprising up to 5% (atomic) of one or more elements selected from the group comprising carbon, nitrogen, arsenic and selenium.
